(19) United States
(12) Patent Application Publication
(10) Pub. No.: US 2025/0032571 A1
(43) Pub. Date: Jan. 30, 2025

(54) Oncidium plant extract and the use for the manufacture of a drug for treating diabetes mellitus, liver protection, preventing acute respiratory distress syndrome (ARDS) thereof

(71) Applicant: Chang Gung University of Science and Technology, Taoyuan City (TW)

(72) Inventors: Tsong-Long Hwang, Taoyuan City (TW); Yu-Chia Chang, Taoyuan City (TW)

(73) Assignee: Chang Gung University of Science and Technology, Taoyuan City (TW)

(21) Appl. No.: 18/784,967

(22) Filed: Jul. 26, 2024

Related U.S. Application Data
(60) Provisional application No. 63/516,145, filed on Jul. 28, 2023.

(51) Int. Cl. A61K 36/898; A61P 1/16; A61P 3/10; A61P 11/00

(52) U.S. Cl. CPC A61K 36/898; A61P 1/16; A61P 3/10; A61P 11/00; A61K 2236/15; A61K 2236/17; A61K 2236/333; A61K 2236/39

(57) ABSTRACT

An Oncidium plant extract and methods for treating diabetes mellitus, liver protection and preventing acute respiratory distress syndrome thereof are provided, the steps for extracting the Oncidium plant extract include taking an Oncidium plant sample, drying and grinding the sample to obtain a ground Oncidium plant sample, and then extracting the ground Oncidium plant sample in a solvent by an accelerated solvent extraction or an ultrasonic extraction to obtain an Oncidium plant extract.

### CROSS-REFERENCE TO RELATED APPLICATION

This application claims the benefit of U.S. Provisional Patent Application No. 63/516,145 filed on Jul. 28, 2023, which is incorporated by reference herein in its entirety.

Oncidium plant extract and the use for the manufacture of a drug for treating diabetes mellitus, liver protection, preventing acute respiratory distress syndrome (ARDS) thereof

### FIELD OF THE INVENTION

The present invention is related to an Oncidium plant extract and the method for treating diabetes mellitus, liver protection, preventing acute respiratory distress syndrome (ARDS) thereof.

### BACKGROUND OF THE INVENTION

Diabetes mellitus (DM) is a metabolic disease caused by impairment of insulin and/or insulin production, which leads to hyperglycemia. Patients with DM often have a number of complications, one of which is impairment of self-healing.

Diabetic wound healing disorder is the main and most intractable complication of diabetic patients, which is related to a variety of factors such as blood vessels, neuropathy, immune and biochemical components. Hyperglycemia hardens the arteries, which in turn leads to slowing of circulation and microvascular dysfunction, as well as reduced tissue oxygenation; Hyperglycemia also reduces the migration of white blood cells to the wound, making people more susceptible to infection. Peripheral neuropathy in DM may cause numbness in the area and a reduced ability to feel pain, which may lead to the wound not being noticed immediately and appropriate treatment to be taken.

Liver is one of the most important organs in the human body, it can be damaged by drugs, alcohol, diet, and poor work and rest. Liver injury can lead to degeneration, necrosis, fibrous tissue hyperplasia and other lesions of hepatocytes, which in turn will lead to a series of liver pathological changes and secondary diseases, such as hepatitis, liver cancer, and liver cirrhosis.

Glutamic Oxaloacetic Transaminase (GOT) and Glutamic Pyruvic Transaminase (GPT) are mainly responsible for the metabolism of amino acids and proteins in the human body, and because of its high concentration in liver cells, once the liver is damaged, GOT and GPT will flow into the blood, so they are used as biological indexes to identify liver injury in blood tests.

Acute respiratory distress syndrome (ARDS) is an acute inflammatory injury to the lungs, which affects the normal function of the alveoli and affects respiratory exchange, resulting in insufficient oxygen concentration in the blood, and successive damage to various organs of the body, which is a fatal acute illness. The treatment of ARDS is based on supportive care such as artificial respirators.

Natural compounds are advantageous for their abundant supplies and diverse skeletons, and they are important bases for drug development. From 1981 till 2019, nearly half of the new FDA-approved drugs were derived from natural products or their derivatives, for example, cocaine-derived narcotics, morphine-derived analgesics, vincristine, doxorubicin and paclitaxel for treating cancers, and fungus-derived penicillin as antibiotics. Therefore, the present invention actively studies to determine which natural compounds have the potential to be developed as novel drugs for treating DM, liver protection, or preventing ARDS.

### SUMMARY OF THE INVENTION

The present invention is an Oncidium plant extract, its preparation steps are to take an Oncidium plant sample, dry and grind the sample to obtain a ground Oncidium plant sample, and then extract the ground Oncidium plant sample in a solvent by an accelerated solvent extraction or an ultrasonic extraction to obtain an Oncidium plant extract.

In the present invention, the solvent is preferably alcohols, esters, or water, and the Oncidium plant extract preferably comprises alcohols extract, esters extract, or an extraction that is extracted by water and then extracted by esters of the Oncidium plant.

The present invention is related to a method for treating diabetes mellitus, comprising administering an effective amount of Oncidium plant extract to a subject suffering from diabetes mellitus.

The present invention is related to a method for liver protection, comprising administering an Oncidium plant extract to a subject in need thereof.

The present invention is related to a method for preventing acute respiratory distress syndrome (ARDS), comprising administering an Oncidium plant extract to a subject suffering from ARDS.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the efficacy of each Oncidium plant extract in inhibiting nitrogen free radical DPPH. **/*** means significantly different.

FIG. 2 illustrates the efficacy of each Oncidium plant extract in inhibiting oxygen free radical AAPH. AAUC means area under the curve of receiver operating characteristic curve; **/*** means significantly different.

FIG. 3 shows the flow chart of animal experiment for evaluating the efficacy of Oncidium plant extract in treatment of type I diabetes mellitus. Symbol description: * indicates induction of diabetes mellitus by streptozotocin; $ indicates checking non-fasting blood glucose level; #indicates checking 18-hours fasting blood glucose level; →indicates the periods for administering Oncidium plant extract; ! indicates sacrifice; % indicates blood parameter to determine the liver function indexes (Glutamic Oxaloacetic Transaminase (GOT), Glutamic Pyruvic Transaminase (GPT)) and kidney function indexes (creatinine (CRE), urea nitrogen (BUN)).

FIG. 4 illustrates the blood glucose levels before fasting and 18-hours after fasting in healthy mice, diabetic mice, and diabetic mice administered with Oncidium plant extract. * indicates significant difference (P<0.05) when treated group is compared to untreated group; +indicates administration; —indicates non-administration.

FIG. 5 illustrates the glucose tolerance test in healthy mice, diabetic mice, and diabetic mice administered with Oncidium plant extract. * indicates significant difference (P<0.05) when low-dosage ORC001R-U-EtOH group is compared to untreated group; #indicates significant difference (P<0.05) when middle-dosage ORC001R-U-EtOH group is compared to untreated group.

FIG. 6 illustrates the blood parameters of healthy mice, diabetic mice, and diabetic mice administered with Oncidium plant extract. * indicates significant difference when treated group is compared to untreated group; +indicates administration; —indicates non-administration.

FIG. 7 illustrates the staining of liver and kidney sections from healthy mice, diabetic mice, and diabetic mice administered with Oncidium plant extract. +indicates administration; —indicates non-administration.

FIG. 8 illustrates the changes in body weight of healthy mice, diabetic mice, and diabetic mice administered with Oncidium plant extract. * indicates significant difference (P<0.05) when low-dosage ORC001R-U-EtOH group is compared to untreated group; #indicates significant difference (P<0.05) when middle-dosage ORC001R-U-EtOH group is compared to untreated group; @indicates significant difference (P<0.05) when high-dosage ORC001R-U-EtOH group is compared to untreated group.

FIG. 9 shows the flow chart of animal experiment for evaluating the efficacy of Oncidium plant extract in treatment of type 2 diabetes mellitus (T2DM). Symbol description: * indicates checking non-fasting blood glucose level; $ indicates checking 8-hours fasting blood glucose level; #indicates intraperitoneal (I.P.) insulin tolerance test; @ indicates I.P. glucose tolerance test →indicates the periods for administering Oncidium plant extract; ! indicates sacrifice; % indicates blood parameter to determine the liver function indexes (Glutamic Oxaloacetic Transaminase (GOT), Glutamic Pyruvic Transaminase (GPT)) and kidney function indexes (creatinine (CRE), urea nitrogen (BUN)).

FIG. 10 shows the blood glucose levels after 8-hours fasting in T2DM mice, and T2DM mice administered with Oncidium plant extract for four weeks. * * indicates significant difference (P<0.01) when treated group is compared to untreated group.

FIG. 11 shows the glucose tolerance test in T2DM mice, and T2DM mice administered with Oncidium plant extract for four weeks. * indicates significant difference (P<0.01) when 50 mg/kg treated group is compared to untreated group.

FIG. 12 shows the insulin tolerance test in T2DM mice, and T2DM mice administered with Oncidium plant extract for four weeks. #indicates significant difference (P<0.01) when 10 mg/kg treated group is compared to untreated group. @ indicates significant difference (P<0.01) when 25 mg/kg treated group is compared to untreated group. * indicates significant difference (P<0.01) when 50 mg/kg treated group is compared to untreated group.

FIG. 13 shows the blood parameters of T2DM mice, and T2DM mice administered with Oncidium plant extract for four weeks. —indicates non-administration.

FIG. 14 shows the flow chart of animal experiment for evaluating the efficacy of Oncidium plant extract in liver protection. Symbol description: * indicates induction of liver fibrosis by CCl₄; % indicates collecting blood; indicates the periods for administering Oncidium plant extract; ! indicates sacrifice.

FIG. 15 illustrates the week 7 blood biochemical analysis results (GOT, GPT) of rats with liver fibrosis in different groups. +indicates administration; —indicates non-administration.

FIG. 16 illustrates the weekly changes in blood biochemical analysis results of rats with liver fibrosis in different groups.

FIG. 17 illustrates the rats body weight tracking results of rats with liver fibrosis in different groups.

FIG. 18 shows the Oncidium plant extract mitigated D-GalN/LPS-induced acute liver injury (ALI) in mice. +indicates administration; —indicates non-administration. * indicates significant difference (P<0.05) when treated group is compared to untreated group; * * * indicates significant difference (P<0.005) when treated group is compared to untreated group.

FIG. 19 shows the flow chart of animal experiment for evaluating the efficacy of Oncidium plant extract in preventing acute respiratory distress syndrome (ARDS). Symbol description: * indicates induction of ARDS by Lipopolysaccharides (LPS); % indicates administration of Oncidium plant extract; ! indicates sacrifice.

FIG. 20 illustrates the staining results of lung tissue sections in different groups mouse. +indicates administration; —indicates non-administration.

FIG. 21 illustrates the blood analysis results in different groups mouse. +indicates administration; —indicates non-administration.

### DETAILED DESCRIPTION OF THE INVENTION

The present invention is an Oncidium plant extract, its preparation steps are to take an Oncidium plant sample, dry and grind the sample to obtain a ground Oncidium plant sample, and then extract the ground Oncidium plant sample in a solvent by an accelerated solvent extraction or an ultrasonic extraction to obtain an Oncidium plant extract.

In the present invention, the solvent is preferably alcohols, esters, or water, and the Oncidium plant extract preferably comprises alcohols extract, esters extract, or an extraction that is extracted by water and then extracted by esters of the Oncidium plant.

The alcohols of the present invention most preferred comprises but not limits to methanol, ethanol, propanol, isopropanol, butanol, isobutanol, hexanol, the esters most preferred comprises but not limits to methyl acetate, ethyl acetate, butyl acetate.

The Oncidium plant extract of the present invention is preferably extracted from the root, the stem or the leaf of the Oncidium plant, or the mixture composed of the root, the stem and the leaf of the Oncidium plant.

The Oncidium plant extract of the present invention is most preferred extracted from the root of the Oncidium plant.

In the present invention, the species of Oncidium comprises Oncidium stacyi, Oncidium splendidum, Oncidium pusillum, Oncidium jonesianum, Oncidium hawkesiana, Oncidium ascendens, Oncidium ampliatum, or Oncidium flexuosum.

The present invention is related to a method for treating diabetes mellitus, comprising administering an Oncidium plant extract to a subject suffering from diabetes mellitus.

The present invention is also related to a method for liver protection, comprising administering an Oncidium plant extract to a subject in need thereof.

As used herein, the term “liver protection” in the present invention refers to the protection of a subject from liver injury, reducing the probability of liver injury, or alleviating the degree of liver injury. The term “liver injury” of the present invention includes, but is not limited to, liver injury, liver diseases such as hepatitis (including but not limited to alcoholic hepatitis, drug-induced hepatitis, steatohepatitis, chronic hepatitis), liver cancer, liver cirrhosis, liver fibrosis, fatty liver, hepatic decline, and other liver diseases caused by liver injury (including but not limited to acute liver injury or chronic liver injury).

The present invention is further related to a method for preventing acute respiratory distress syndrome (ARDS), comprising administering an Oncidium plant extract to a subject suffering from ARDS.

As used herein, the term “acute respiratory distress syndrome” of the present invention comprises transfusion-related lung injury, ventilator-induced lung injury, bacteria-induced lung injury, or viruses-induced lung injury.

In the present invention, the drug can further comprise a pharmaceutically acceptable carrier for the Oncidium plant extract.

The term “effective amounts” is the amount that can achieve effective results when administered to an individual, or that has the desired activity in vivo or in vitro. In the case of inflammatory disorders, as compared to no treatment, effective clinical outcomes include amelioration of the extent or severity of the symptoms associated with the disease or condition, and/or prolonging the life of an individual and/or improvement of the quality of life of the individual. The exact amount of compound administered to an individual will depend on the type and severity of the disease or symptoms and on the individual characteristics such as the general health of the individual, age, sex, weight, and drug tolerance of the individual. It is also dictated by the conditions, severity and types of the inflammatory disorder, the autoimmune disorder and the allergic disorder, or the desired immunosuppressive effect. Those skilled in the art based on the disclosed technical features of the present invention, will be able to determine the appropriate dose according to these and other factors.

In the present invention, the effective amount of the Oncidium plant extract of the present invention is 0.001 mg/kg-body weight to 100 mg/kg-body weight.

Furthermore, the effective amount of the Oncidium plant extract of the present invention can be 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 25, 26, 27, 28, 29, 30, 31, 32, 33, 34, 35, 36, 37, 38, 39, 40, 41, 42, 43, 44, 45, 46, 47, 48, 50, 51, 52, 53, 54, 55, 56, 57, 58, 59, 60, 61, 62, 63, 64, 65, 66, 67, 68, 69, 70, 71, 72, 73, 75, 76, 77, 78, 79, 80, 81, 82, 83, 84, 85, 86, 87, 88, 89, 90, 91, 92, 93, 94, 95, 96, 97, 98, 100 mg/kg-body weight.

The effective amount of the present invention can be conversed to equivalent effective amount of different species subject according to the formula and the safety factor provided in Guidance for Industry: Establishing the Maximum Safe Starting Doses in Initial Clinical Trials for Therapeutics in Adult Healthy Volunteers, U.S. Department of Health and Human Services, Food and Drug Administration Center for Drug Evaluation and Research (CDER), July 2005 published by FDA.

In the method for treating diabetes mellitus in a subject, the subject is human or mammal, the effective amount of the present invention is 0.007 mg/kg-body weight to 1.5 mg/kg-body weight; the effective amount of the present invention is preferably 0.035 mg/kg-body weight to 1.3 mg/kg-body weight; the effective amount of the present invention is most preferred 0.07 mg/kg-body weight to 0.75 mg/kg-body weight.

In detail, in the method for treating diabetes mellitus in a subject, the effective amount in mouse with average weight of 25 grams of the present invention is 1 mg/kg-body weight to 100 mg/kg-body weight, equivalent to the effective amount in dogs or cats with average weight of 10 kilograms is 0.01 mg/kg-body weight to 1.5 mg/kg-body weight, equivalent to the effective amount in adults with average weight of 60 kilograms is 0.007 mg/kg-body weight to 0.8 mg/kg-body weight, equivalent to the effective amount in children with average weight of 20 kilograms is 0.01 mg/kg-body weight to 1.2 mg/kg-body weight; Preferably, the effective amount in mouse with average weight of 25 grams of the present invention is 5 mg/kg-body weight to 75 mg/kg-body weight, equivalent to the effective amount in dogs or cats with average weight of 10 kilograms is 0.05 mg/kg-body weight to 1.3 mg/kg-body weight, equivalent to the effective amount in adults with average weight of 60 kilograms is 0.035 mg/kg-body weight to 0.6 mg/kg-body weight, equivalent to the effective amount in children with average weight of 20 kilograms is 0.05 mg/kg-body weight to 0.85 mg/kg-body weight; Most preferred, the effective amount in mouse with average weight of 25 grams of the present invention is 10 mg/kg-body weight to 50 mg/kg-body weight, equivalent to the effective amount in dogs or cats with average weight of 10 kilograms is 0.13 mg/kg-body weight to 0.75 mg/kg-body weight, equivalent to the effective amount in adults with average weight of 60 kilograms is 0.07 mg/kg-body weight to 0.45 mg/kg-body weight, equivalent to the effective amount in children with average weight of 20 kilograms is 0.1 mg/kg-body weight to 0.6 mg/kg-body weight.

In the method for liver protection in a subject, the subject is human or mammal, the effective amount of the present invention is 0.001 mg/kg-body weight to 1.75 mg/kg-body weight; the effective amount of the present invention is preferably 0.009 mg/kg-body weight to 1 mg/kg-body weight; the effective amount of the present invention is most preferred 0.015 mg/kg-body weight to 0.4 mg/kg-body weight.

In detail, in the method for liver protection in a subject, the effective amount in rats with average weight of 350 grams of the present invention is 0.1 mg/kg-body weight to 50 mg/kg-body weight, equivalent to the effective amount in dogs or cats with average weight of 10 kilograms is 0.003 mg/kg-body weight to 1.75 mg/kg-body weight, equivalent to the effective amount in adults with average weight of 60 kilograms is 0.001 mg/kg-body weight to 1.25 mg/kg-body weight, equivalent to the effective amount in children with average weight of 20 kilograms is 0.002 mg/kg-body weight to 1.5 mg/kg-body weight; Preferably, the effective amount in rats with average weight of 350 grams of the present invention is 0.5 mg/kg-body weight to 25 mg/kg-body weight, equivalent to the effective amount in dogs or cats with average weight of 10 kilograms is 0.015 mg/kg-body weight to 1 mg/kg-body weight, equivalent to the effective amount in adults with average weight of 60 kilograms is 0.009 mg/kg-body weight to 0.5 mg/kg-body weight, equivalent to the effective amount in children with average weight of 20 kilograms is 0.013 mg/kg-body weight to 0.7 mg/kg-body weight; Most preferred, the effective amount in rats with average weight of 350 grams of the present invention is 1 mg/kg-body weight to 10 mg/kg-body weight, equivalent to the effective amount in dogs or cats with average weight of 10 kilograms is 0.03 mg/kg-body weight to 0.4 mg/kg-body weight, equivalent to the effective amount in adults with average weight of 60 kilograms is 0.015 mg/kg-body weight to 0.2 mg/kg-body weight, equivalent to the effective amount in children with average weight of 20 kilograms is 0.025 mg/kg-body weight to 0.3 mg/kg-body weight.

In the method for preventing acute respiratory distress syndrome (ARDS) in a subject, the subject is human or mammal, the effective amount of the present invention is 0.007 mg/kg-body weight to 0.75 mg/kg-body weight; the effective amount of the present invention is preferably 0.02 mg/kg-body weight to 0.4 mg/kg-body weight; the effective amount of the present invention is most preferred 0.035 mg/kg-body weight to 0.35 mg/kg-body weight.

In detail, in the method for preventing acute respiratory distress syndrome (ARDS) in a subject, the effective amount in mouse with average weight of 25 grams of the present invention is 1 mg/kg-body weight to 50 mg/kg-body weight, equivalent to the effective amount in dogs or cats with average weight of 10 kilograms is 0.01 mg/kg-body weight to 0.75 mg/kg-body weight, equivalent to the effective amount in adults with average weight of 60 kilograms is 0.007 mg/kg-body weight to 0.45 mg/kg-body weight, equivalent to the effective amount in children with average weight of 20 kilograms is 0.01 mg/kg-body weight to 0.65 mg/kg-body weight; Preferably, the effective amount in mouse with average weight of 25 grams of the present invention is 3 mg/kg-body weight to 25 mg/kg-body weight, equivalent to the effective amount in dogs or cats with average weight of 10 kilograms is 0.04 mg/kg-body weight to 0.4 mg/kg-body weight, equivalent to the effective amount in adults with average weight of 60 kilograms is 0.02 mg/kg-body weight to 0.25 mg/kg-body weight, equivalent to the effective amount in children with average weight of 20 kilograms is 0.03 mg/kg-body weight to 0.3 mg/kg-body weight; Most preferred, the effective amount in mouse with average weight of 25 grams of the present invention is 5 mg/kg-body weight to 20 mg/kg-body weight, equivalent to the effective amount in dogs or cats with average weight of 10 kilograms is 0.06 mg/kg-body weight to 0.35 mg/kg-body weight, equivalent to the effective amount in adults with average weight of 60 kilograms is 0.035 mg/kg-body weight to 0.2 mg/kg-body weight, equivalent to the effective amount in children with average weight of 20 kilograms is 0.05 mg/kg-body weight to 0.25 mg/kg-body weight.

In the present invention, the subject of the present invention is human (e.g. infant, toddler, teenager, young adult, middle-aged adult or elderly), rodent (e.g. mice, rat) or mammal (e.g. companion animal such as dog, cat, rabbit, marten; economic animals such as horse, sheep, cow, pig). In a preferred manner, the subject of the present invention is human or mammal. The subject of the present invention is most preferred human.

The present can also comprise a pharmaceutically acceptable carrier, particularly can further comprise predetermined solvents or oils, PH adjuster and if desired, can further comprise a dispersant. Examples of solvents used in the present invention include, but are not limited to, water, ethanol, isopropanol, 1,3-butanediol, propylene glycol, glycerin, etc. Examples of oils used in the present invention are selected from the group consisting of, but are not limited to, corn oil, sesame oil, flaxseed oil, cottonseed oil, soybean oil, peanut oil, mono-glycerides, di-glycerides, tri-glycerides, mineral oil, squalene, jojoba oil, olive oil, evening primrose oil, borage oil, grape seed oil, coconut oil, sunflower oil, shea butter, and any combinations thereof.

Solvents and oils can be used alone or in any combinations thereof.

Examples of useful dispersants that are beneficial to the present invention can include, but are not limited to, lecithin, organic monoglycerides, sorbitan fatty acid esters, polyoxyethylene fatty acid esters, sorbitan stearate, etc. These raw materials can also be used alone or in any combinations thereof.

In the preset invention, the drug is preferably administered orally or by injection.

In the present invention, the drug of the present invention can also be prepared as external preparations, examples of the external preparations can include, but are not limited to, creams, ointments, gels, wash lotions or patches, etc., or inhalants, aerosols, suppositories, etc.

When the drug is used as an external preparation, an appropriate external skin preparation can be used as a base material, and an aqueous solution, a non-aqueous solvent, a suspension, an emulsion or a lyophilized preparation, etc., can be used and sterilized according to known methods. The compositions in the form of gels, creams and ointments can be prepared according to the form of the composition by using known methods, and by addition of known softeners, emulsifiers and thickeners or other materials known in the art.

The gel-form composition can be prepared, for example, by addition of a softener such as trimethylolpropane, polyethylene glycol and glycerol, for example, a solvent of propylene glycol, ethanol and isocetyl alcohol, and pure water.

### Description of Embodiments

The present embodiment only describes the best example but is not intended to limit the present invention.

### Preparation of Oncidium Plant Extract

In the present invention, the Oncidium plant sample used for extraction preparation was either the root (R), stem (B) or leaf (L) of Oncidium plant, or a mixture of its root, stem and leaf. The species of Oncidium plant could be selected from Oncidium stacyi, Oncidium splendidum, Oncidium pusillum, Oncidium jonesianum, Oncidium hawkesiana, Oncidium ascendens, Oncidium ampliatum, or Oncidiumflexuosum. The Oncidium plant sample was dried and ground and stored at −20° C. until it was ready for use.

In the present invention, the Oncidium sample was first extracted by accelerated solvent extraction (A) and ultrasonic extraction (U).

In the accelerated solvent extraction, ethyl acetate (A-EA), 95% ethanol (A-EtOH), H₂O (A-W), and the residue after extracted with H₂O then was re-extracted by using ethyl acetate (A-W+EA) were used as solvent for purification and extraction, respectively, the extracted temperature was 30˜70° C. (preferably was 50° C.), the extracted time was 10˜30 minutes (preferably was 15 minutes), the extracted pressure was 1300˜1700 psi (since the slight pressure fluctuation during the extraction process, preferably was average pressure of 1500 psi), and then obtained Oncidium plant extracts (ORC001) of the present invention. The extractions were named according to the different extraction portion and the different solvent: ORC001R-A-EA, ORC001R-A-EtOH, ORC001R-A-W, ORC001R-A-W+EA, ORC001B-A-EA, ORC001B-A-EtOH, ORC001B-A-W, ORC001B-A-W+EA, ORC001L-A-EA, ORC001L-A-EtOH, ORC001L-A-W, and ORC001L-A-W+EA.

In the ultrasonic extraction, ethyl acetate (U-EA), 95% ethanol (U-EtOH), H₂O (U-W), and the residue after extracted with H₂O then was re-extracted by using ethyl acetate (U-W+EA) were used as solvent for purification and extraction at room temperature, respectively, the extracted temperature was 30˜70° C. (preferably was 50° C.), the extracted time was 15˜60 minutes (preferably was 30 minutes), the output power was 200˜600 watts (preferably was 400 watts), the frequency was 20˜60 Hzs (preferably was 40 Hzs), and then obtained Oncidium plant extracts (ORC001) of the present invention. The extractions were named according to the extraction portion and the solvent: ORC001R-U-EA, ORC001R-U-EtOH, ORC001R-U-W, ORC001R-U-W+EA, ORC001B-U-EA, ORC001B-U-EtOH, ORC001B-U-W, ORC001B-U-W+EA, ORC001L-U-EA, ORC001L-U-EtOH, ORC001L-U-W, and ORC001L-U-W+EA.

Determination of the components profiles of the different species of Oncidium plant extracts (same portion, same solvent, same extraction method) were carried out by high performance liquid chromatography (HPLC) fingerprint, the results revealed that the similar fingerprints were observed in the different species of Oncidium plant extracts, which indicated that same compounds exist in those different species of Oncidium plant extracts. The species Oncidium flexuosum was therefore selected as the Oncidium plant sample of the present invention.

### Inhibition Test of Superoxide Anion and Elastase Release

The anti-inflammatory ability of the above-mentioned various Oncidium plant extracts were evaluated in the present invention through inhibition test of superoxide anion and elastase release.

First, the preparation of neutrophils was carried out.

Healthy donors (aged 20-35; normal work and rest and abstaining from taking drugs for more than one week), about 50 mL of blood was collected from the elbow vein with a vacuum sterile blood collection tube, which was added to an equal volume of 3% dextran solution (Dextran) and mixed evenly, and allowed to stand to settle red blood cells. The supernatant containing neutrophils was taken and covered it on Ficoll-Paque solution which was contained in a centrifuge tube, then centrifuged the centrifuge tube, and different blood cells were separated by density gradient to obtain neutrophils and a few red blood cells precipitated at the bottom. The residual red blood cells were expanded with different concentrations of NaCl solution, and the required neutrophils were retained by taking advantage of the difference in osmotic pressure tolerance between the two cells.

The evaluation of the inflammatory response of the above-mentioned various Oncidium plant extracts against neutrophils were carried out by the respiratory blasting experiment and the evaluation of the degranulation function.

Respiratory blasting mainly produces superoxide anions (O₂^{•-}) and reactive oxygen species (ROS), the detection of superoxide anion (O₂^{•-}) was performed by adding a neutrophil suspension (6×10⁵ cells/mL) containing 0.6 mg/mL of ferricytochrome c to the above Oncidium plant extracts at 37° C. for 2 minutes, and then treated with 1 μg/mL cytochalasin B (CB) for 3 minutes. Then, N-formyl-L-methionyl-L-leucinoyl-L-phenylalanine (fMLF) was used as a stimulant to activate the cells for about 10 minutes. The absorbance value was then measured at a wavelength of 550 nm using a UV spectrophotometer.

In the elastase release experiment, a neutrophil suspension (6×10⁵ cells/mL) containing 100 μM elastase acceptor methoxysuccinyl-ala-ala-prolineate-valine-p-nitroanilide (MeOSuc-Ala-Ala-Pro-Val-p-nitroanilide) was pre-warmed for 5 minutes to reach 37° C., followed by the addition of the above various Oncidium plant extracts for 2 minutes. Cells were treated with 0.5 μg/mL cytochalasin B (CB) for 3 minutes, the neutrophils were activated by stimulation of 0.1 μM fMLF for approximately 10 minutes, and the absorbance value was finally measured at 405 nm using a UV spectrophotometer. Experimental results were expressed as percentage inhibition.

The results were shown in table 1.

TABLE 1

| | Superoxide anion | | | Elastase release | | |
|---|---|---|---|---|---|---|
| | IC₅₀ | | | IC₅₀ | | |
| Extract | (μg/mL) ^{a} | Inh % | | (μg/mL) ^{a} | Inh % | |
| ORC001R-A-EA^{c} | 0.62 ± 0.13 | 94.78 ± 0.46 | *** | 0.75 ± 0.06 | 120.09 ± 2.14 | *** |
| ORC001R-A-EtOH | 1.08 ± 0.16 | 98.94 ± 0.56 | *** | 1.25 ± 0.33 | 110.71 ± 1.37 | *** |
| ORC001R-A-W^{b} | >10 | 39.90 ± 9.84 | ** | >10 | 21.58 ± 7.30 | * |
| ORC001R-A-W + EA | 0.62 ± 0.06 | 95.00 ± 0.62 | *** | 0.87 ± 0.14 | 116.19 ± 2.59 | *** |
| ORC001R-U-EA | 0.51 ± 0.11 | 94.34 ± 0.83 | *** | 0.54 ± 0.09 | 107.07 ± 4.50 | *** |
| ORC001R-U-EtOH | 0.95 ± 0.22 | 101.34 ± 0.48 | *** | 1.61 ± 0.59 | 111.72 ± 9.08 | *** |
| ORC001R-U-W^{b} | >10 | 18.31 ± 8.40 | | >10 | 20.93 ± 4.62 | * |
| ORC001R-U-W + EA | 0.55 ± 0.17 | 94.52 ± 0.82 | *** | 0.63 ± 0.19 | 115.12 ± 7.79 | *** |
| ORC001B-A-EA | 4.00 ± 0.81 | 91.52 ± 1.54 | *** | 3.75 ± 0.41 | 89.10 ± 7.18 | *** |
| ORC001B-A-EtOH | 5.58 ± 0.86 | 72.34 ± 5.47 | *** | 4.82 ± 0.98 | 71.43 ± 7.65 | *** |
| ORC001B-A-W^{b} | >10 | 43.85 ± 4.01 | *** | >10 | 35.54 ± 8.42 | * |
| ORC001B-A-W + EA | 3.47 ± 0.73 | 98.23 ± 0.71 | *** | 2.99 ± 0.20 | 107.73 ± 6.06 | *** |
| ORC001B-U-EA | 3.76 ± 0.78 | 88.56 ± 1.23 | *** | 3.33 ± 0.53 | 95.71 ± 2.22 | *** |
| ORC001B-U-EtOH | 5.28 ± 0.88 | 76.39 ± 4.76 | *** | 4.92 ± 0.07 | 81.53 ± 4.34 | *** |
| ORC001B-U-W^{b} | >10 | 46.06 ± 2.01 | *** | >10 | 31.10 ± 0.48 | *** |
| ORC001B-U-W + EA | 3.98 ± 0.98 | 88.47 ± 3.42 | *** | 3.27 ± 0.47 | 84.49 ± 9.49 | *** |
| ORC001L-A-EA | 7.52 ± 1.87 | 53.67 ± 6.72 | *** | 6.06 ± 0.66 | 73.72 ± 7.18 | *** |
| ORC001L-A-EtOH | 4.35 ± 0.42 | 92.41 ± 3.30 | *** | 3.51 ± 0.71 | 91.59 ± 8.55 | *** |
| ORC001L-A-W^{b} | >10 | 11.04 ± 5.09 | | >10 | 6.42 ± 7.11 | |
| ORC001L-A-W + EA | 7.11 ± 0.86 | 65.90 ± 4.71 | *** | 5.13 ± 0.78 | 77.65 ± 3.49 | *** |
| ORC001L-U-EA | 7.43 ± 0.56 | 62.11 ± 3.72 | *** | >10 | 46.98 ± 4.23 | *** |
| ORC001L-U-EtOH | 5.49 ± 0.42 | 84.13 ± 4.87 | *** | 5.56 ± 0.32 | 86.92 ± 5.84 | *** |
| ORC001L-U-W^{b} | >10 | 11.94 ± 2.43 | ** | >10 | −8.03 ± 5.11 | |
| ORC001L-U-W + EA | 6.91 ± 0.92 | 66.26 ± 4.76 | *** | >10 | 38.44 ± 7.45 | ** |

Percentage of inhibition (Inh %) at 10 μg/ml. Results are presented as mean ± S.E.M. (n = 3-5).
* P < 0.05,
** P < 0.01,
*** P < 0.001 compared with the control (DMSO or H₂O).
^{a} Concentration necessary for 50% inhibition (IC₅₀).
^{b}Dissolved in H₂O.
^{c}ORC001R-A-EA (10 μg/ml) was filtered with a 0.22 μm filter before the experiment.

The cytotoxicity of the Oncidium plant extracts in the present invention were shown in Table 2.

TABLE 2

| Extract | Cell viability (%) |
|---|---|
| ORC001R-A-EA^{a} | 98.61 ± 0.82 |
| ORC001R-A-EtOH | 95.60 ± 1.99 |
| ORC001R-A-W + EA | 97.84 ± 1.18 |
| ORC001R-U-EA | 97.90 ± 3.43 |
| ORC001R-U-EtOH | 92.35 ± 3.39 |
| ORC001R-U-W + EA | 97.09 ± 1.40 |
| ORC001B-A-EA | 95.24 ± 1.89 |
| ORC001B-A-EtOH | 101.62 ± 1.57 |
| ORC001B-A-W + EA | 100.72 ± 1.72 |
| ORC001B-U-EA | 102.18 ± 1.48 |
| ORC001B-U-EtOH | 101.86 ± 3.57 |
| ORC001B-U-W + EA | 98.21 ± 1.84 |
| ORC001L-A-EA | 101.44 ± 3.63 |
| ORC001L-A-EtOH | 100.19 ± 1.98 |
| ORC001L-A-W + EA | 100.20 ± 0.64 |
| ORC001L-U-EA | 98.89 ± 1.64 |
| ORC001L-U-EtOH | 100.52 ± 0.93 |
| ORC001L-U-W + EA | 99.74 ± 1.51 |

Percentage of cell viability (%) at 10 μg/ml. Results are presented as mean ± S.E.M. (n = 3).
^{a}ORC001R-A-EA (10 μg/ml) was filtered with a 0.22 μm filter before the experiment.

### Free Radical Scavenging Capacity Test

Nitrogen radicals: 1,1-diphenyl-p-picrylhydrazyl (DPPH), and oxygen radicals: 2,2′-Azobis (2-amidinopropane) hydrochloride (AAPH) are stable free radicals associated with inflammation. In the present invention, the ability of 10 μg/mL of each Oncidium plant extract to scavenge free radicals was further tested, wherein DMSO or H₂O was used as control group, α-Tocopherol was used as compared reagent, and when each Oncidium plant extract had the ability to scavenge free radicals, it was mixed with ethanol-configured DPPH (100 μM) or AAPH (25 mM) reaction solution for 30 minutes and 2 hours respectively, respectively, as the activity index of free radical scavenging.

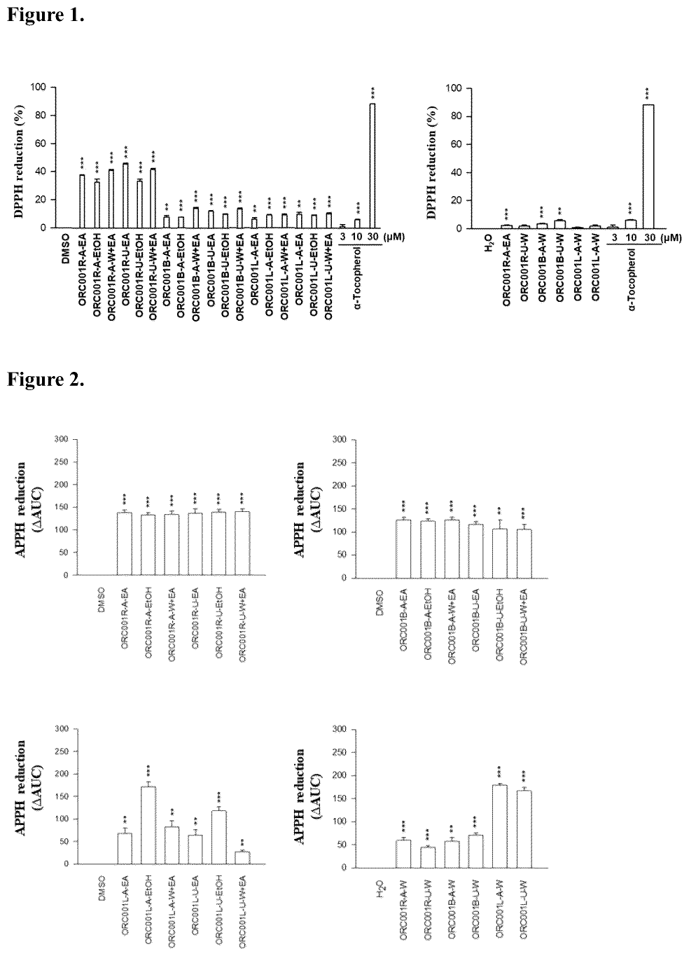

As shown in FIG. 1, the organic solvent extracts (EA, EtOH, W+EA) of Oncidium root (ORC001R) were more significant in the inhibition of nitrogen radical DPPH.

In addition, as shown in FIG. 2, the organic solvent extracts (EA, EtOH, W+EA) of Oncidium root (ORC001R) and Oncidium stem (ORC001B), and the EtOH and water extracts of Oncidium leaf (ORC001L) showed more significant in the inhibition of oxygen radicals.

### Establishment of Diabetes Mellitus Type 1 (T1DM) Model

All experiments were approved by the Chang Gung University Institutional Animal Care and Use Committee (IACUC).

First, the animal models of diabetes mellitus were established. Diabetes mellitus was induced in 7-week-old C57BL/6 male mice weighed 20-25 grams by fasting for 4 hours, thereafter injected intraperitoneal with 55 mg/kg Streptozotocin (STZ) (Sigma-Aldrich) in citrate buffer (pH 4.5) for 5 consecutive days. A normal chow fed and 1% glucose water was provided after STZ injection from day 1 to day 4 for induction of type 1 diabetes. Normal potable water was provided instead of providing glucose water on day 5 onwards.

After 72 hours post STZ injection, mice were fasted for 4 hours. The tail was pricked and bled to measure fasting blood glucose levels using Contour Plus portable glucometer (Ascensia, United Kingdom). Animals with fasting blood glucose level of 150-250 mg/dL were considered pre-diabetic, whereas >250 mg/dL were considered diabetic and used for subsequent experiments. The fasting blood levels of all the mice were >250 mg/dL.

### Evaluation of T1DM Treatment Effect

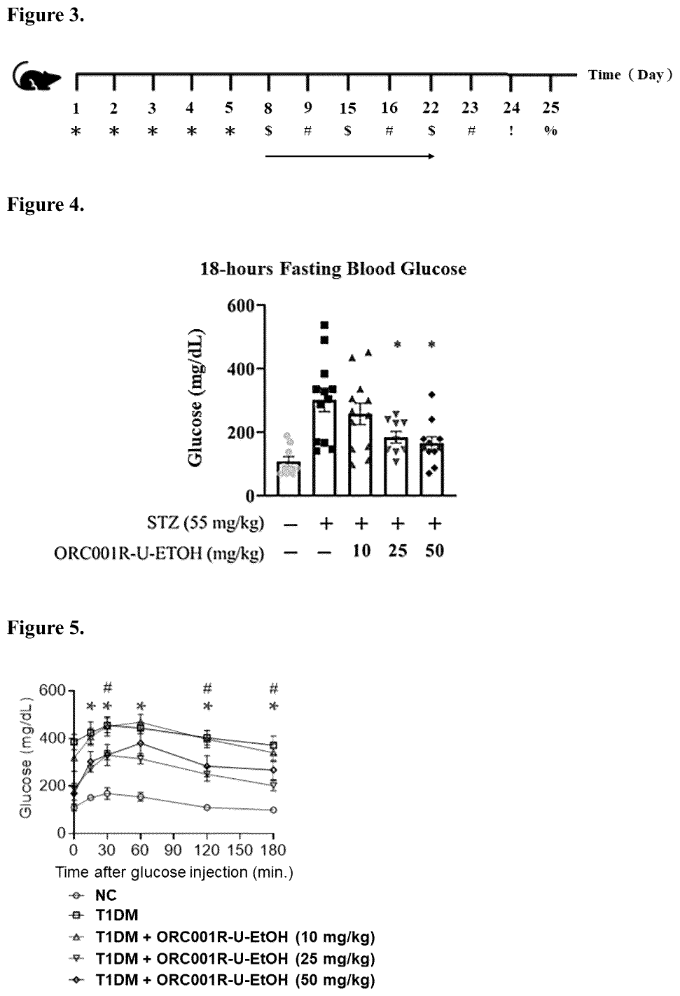

The therapeutic effect of Oncidium root extract (ORC001R) in diabetes was evaluated by monitoring the blood glucose level with or without the extract administration. The Oncidium root ultrasonic extract with EtOH as solvent (ORC001R-U-EtOH) was used for the present invention. The experiment flow chart is shown in FIG. 3.

Total 25 C57BL/6 male mice were used in the experiment. Those mice were randomly divided to 5 groups: 4 mice for treatment control group (10% ethanol and 10% Tween-20 in saline; NC), 7 mice for untreated group (STZ; T1DM), 7 mice for low-dosage ORC001R-U-EtOH group (administering 10 mg/kg ORC001R-U-EtOH; T1DM+ORC001R-U-EtOH (10 mg/kg)), 4 mice for middle-dosage ORC001R-U-EtOH group (administering 25 mg/kg ORC001R-U-EtOH; T1DM+ORC001R-U-EtOH (25 mg/kg)), and 3 mice for high-dosage ORC001R-U-EtOH group (administering 50 mg/kg ORC001R-U-EtOH; T1DM+ORC001R-U-EtOH (50 mg/kg)).

Prior to drug administration, the weight of animals was checked, and the non-fasting blood glucose level of animals were checked by pricked the end of tail and collected the blood by using Contour Plus portable glucometer (Ascensia, United Kingdom). Thereafter, the animals were fasted for 18 hours to obtain fasting blood glucose level. These steps were repeated on the 7th and 14th day post drug administration to observe the trend of blood glucose level.

As shown in FIG. 4, the result of the 18-hours fasting blood level of the animals on 14 days-post administration showed that the low-dosage ORC001R-U-EtOH group showed a tendency to decrease blood glucose, the preferred effect in decreasing blood glucose was observed in middle-dosage ORC001R-U-EtOH group, and a significant effect in decreasing blood glucose was observed in high-dosage ORC001R-U-EtOH group.

The result of trend of blood glucose level of the animals was examed by intraperitoneal glucose tolerance test (IPGTT). Mice were intraperitoneally injected with glucose (purchased from Sigma) at a dose of 1 g/kg body-weight after 18 hours fasting, and the blood glucose level was detected at time points of 0 minute, 15 minutes, 30 minutes, 60 minutes, 120 minutes and 180 minutes after injection by using glucometer. The result is shown in FIG. 5, which demonstrated that all the low-dosage ORC001R-U-EtOH group, middle-dosage ORC001R-U-EtOH group and high-dosage ORC001R-U-EtOH group had the trend of decreasing blood glucose level after orally administration for 14 consecutive days, particularly, the best trends of decreasing blood glucose level were showed in both middle-dosage ORC001R-U-EtOH group and high-dosage ORC001R-U-EtOH group.

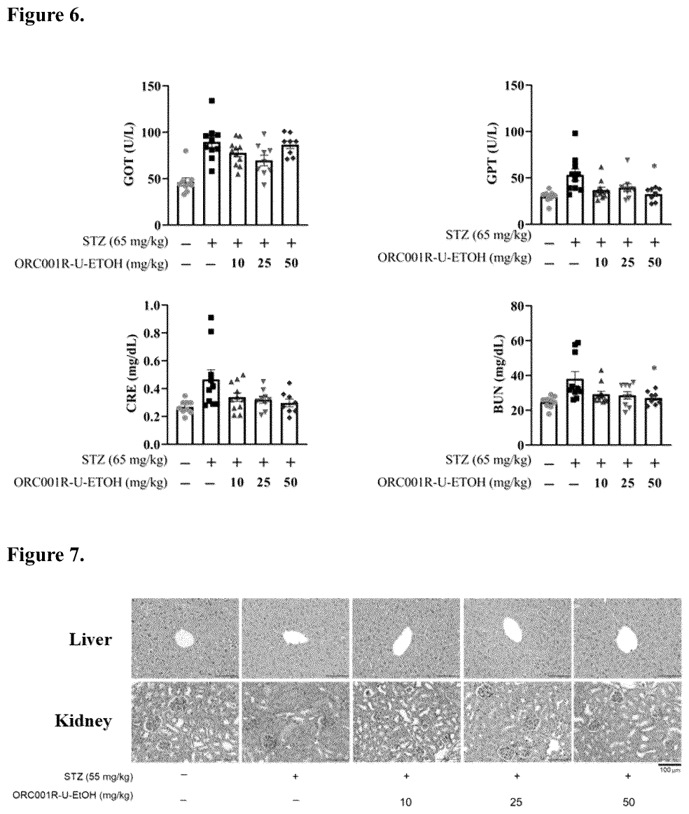

After 14 consecutive days of ORC001R-U-EtOH treatment, blood was drawn from the heart of the animal model and then centrifuged at 2000×g for 15 minutes at room temperature to separate the serum from whole blood, the liver function indexes (Glutamic Oxaloacetic Transaminase (GOT), Glutamic Pyruvic Transaminase (GPT)) and kidney function indexes (creatinine (CRE), urea nitrogen (BUN)) were tested in the blood of the animals by using Fujifilm DRI-CHEM NX500i device, and liver and kidney tissues were also sectioned and stained with hematoxylin-eosin (H&E stain), the results are shown in FIGS. 6 and 7, the ORC001R-U-EtOH did not show toxicity in the liver or kidney, and also had the effect of repairing liver and kidney damage caused by diabetes mellitus.

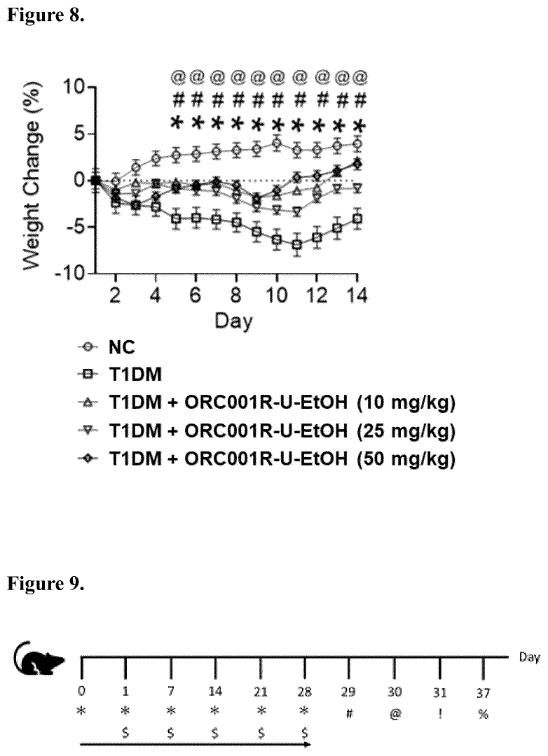

The changes in weight of the animals is shown in FIG. 8, the ORC00IR-U-EtOH administration in animals showed the trend of weight regain from day 9 onwards.

### Establishment of Diabetes Mellitus Type 2 (T2DM) Model

All experiments were approved by the Chang Gung University Institutional Animal Care and Use Committee (IACUC).

Firstly, a type 2 diabetes animal model was established. This invention selected 5-week-old male BKS.Cg-Dock7m+/+Leprdb/Jnarl mice, weighing 31-35 grams. This strain is a genetically modified spontaneous type 2 diabetic mouse, which has been established for many years and is widely accepted as a model for metabolic syndrome diseases.

The tail was pricked and bled to measure fasting blood glucose levels using Contour Plus portable glucometer (Ascensia, United Kingdom). Animals with fasting blood glucose levels of 150-250 mg/dL are considered to be in a pre-diabetic state. As the weeks of the experiment progress, the fasting blood glucose levels of the mice gradually increase, with levels >250 mg/dL being classified as diabetes.

### Evaluation of T2DM Treatment Effect

The therapeutic effect of Oncidium root extract (ORC001R) in diabetes was evaluated by monitoring the blood glucose level with or without the extract administration. The Oncidium root ultrasonic extract with EtOH as solvent (ORC001R-U-EtOH) was used for the present invention. The experiment flow chart is shown in FIG. 9.

Total 12 BKS.Cg-Dock7m+/+Leprdb/Jnarl male mice were used in the experiment. Those mice were randomly divided to 4 groups: 3 mice for untreated group (T2DM) (1% tween 80 and 99% CMC solution (0.5% w/v); T2DM), 3 mice for low-dosage ORC001R-U-EtOH group (administering 10 mg/kg ORC001R-U-EtOH; T2DM+ORC001R-U-EtOH (10 mg/kg)), 3 mice for middle-dosage ORC001R-U-EtOH group (administering 25 mg/kg ORC001R-U-EtOH; T2DM+ORC001R-U-EtOH (25 mg/kg)), and 3 mice for high-dosage ORC001R-U-EtOH group (administering 50 mg/kg ORC001R-U-EtOH; T2DM+ORC001R-U-EtOH (50 mg/kg)).

Prior to drug administration, the weight of animals was checked, and the non-fasting blood glucose level of animals were checked by pricked the end of tail and collected the blood by using Contour Plus portable glucometer (Ascensia, United Kingdom). Thereafter, the animals were fasted for 8 hours to obtain fasting blood glucose level. These steps were repeated on the 1st, 7th, 14th, 21st and 28th day post drug administration to observe the trend of blood glucose level.

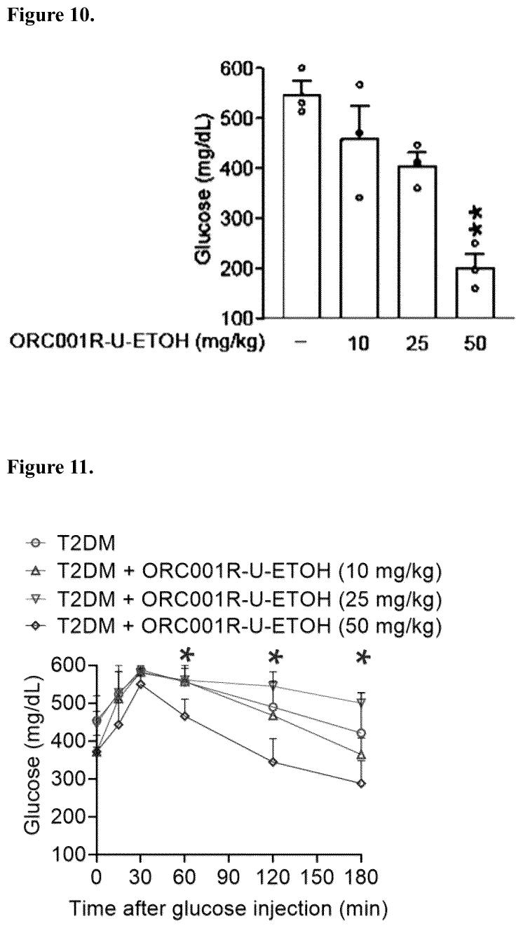

The fasting blood glucose levels of the experimental animals at 28 days after treatment are shown in FIG. 10. The results indicate that the root extract of ORC001R-U-EtOH exhibits a trend of lowering blood glucose at low doses. At medium doses, it shows a more pronounced hypoglycemic effect, and at high doses, it demonstrates a significant hypoglycemic effect, exhibiting a dose-dependent response.

The result of trend of blood glucose level of the animals was examined by intraperitoneal glucose tolerance test (IPGTT). Mice were intraperitoneally injected with glucose (purchased from Sigma) at a dose of 1 g/kg body-weight after 18 hours fasting, and the blood glucose level was detected at time points of 0 minute, 15 minutes, 30 minutes, 60 minutes, 120 minutes and 180 minutes after injection by using glucometer. The result is shown in FIG. 11, which demonstrated that all the low-dosage ORC001R-U-EtOH group, middle-dosage ORC001R-U-EtOH group and high-dosage ORC001R-U-EtOH group had the trend of decreasing blood glucose level after orally administration for 28 consecutive days, particularly, the best trends of decreasing blood glucose level were showed in both low-dosage ORC001R-U-EtOH group and high-dosage ORC001R-U-EtOH group. Only the high dose shows a significant improvement when compared to the untreated group.

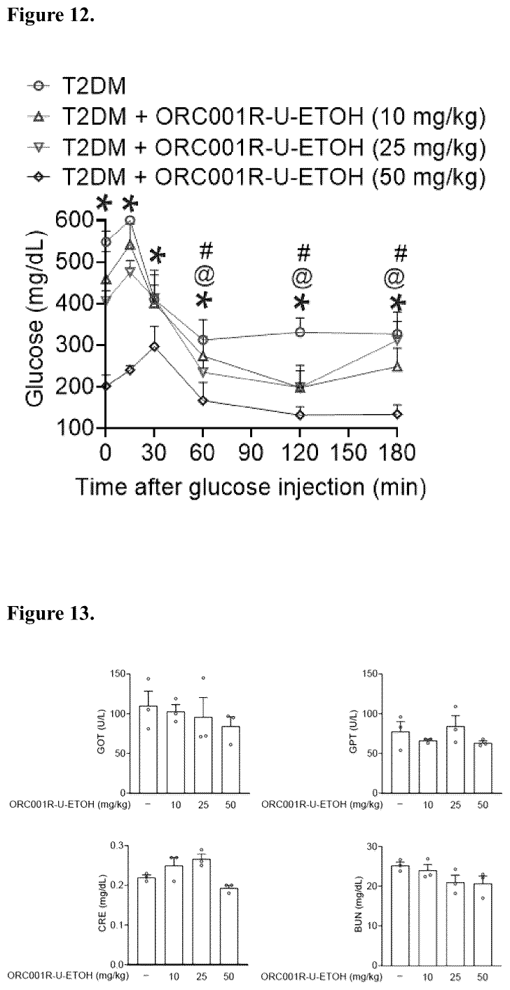

For I.P. Insulin Tolerance Tests (IPITT), mice were injected with insulin (Humulin R U-500, Lilly) 0.75 U/kg body weight after 5 hours fasting, and glucose concentration was determined with the Glucometer at time points 0 minute, 15 minutes, 30 minutes, 60 minutes, and 120 minutes. The result is shown in FIG. 12, which demonstrated that all the low-dosage ORC001R-U-EtOH group, middle-dosage ORC001R-U-EtOH group and high-dosage ORC001R-U-EtOH group had the trend of decreasing blood glucose level after orally administration for 28 consecutive days. This trend demonstrates a significant improvement in improving insulin sensitivity, exhibiting a dose-dependent response.

After 28 consecutive days of ORC001R-U-EtOH treatment, blood was drawn from the heart of the animal model and then centrifuged at 2000×g for 15 minutes at room temperature to separate the serum from whole blood, the liver function indexes (Glutamic Oxaloacetic Transaminase (GOT), Glutamic Pyruvic Transaminase (GPT)) and kidney function indexes (creatinine (CRE), urea nitrogen (BUN)) were tested in the blood of the animals by using Fujifilm DRI-CHEM NX500i device. The results are shown in FIG. 13. The ORC001R-U-EtOH did not show toxicity in the liver or kidney.

### Evaluating the Effect for Liver Protection

All experiments on animals complied with Chang Gung University's IACUC guidelines.

The experimental method was designed with reference to the evaluation method of liver protection and health care efficacy of health food of the Ministry of Health and Welfare of Taiwan (revised version of the Announcement Food Characters No. 1031304063 of the Department of Health on Apr. 25, 105).

First, the CCl₄ solution and ORC001R-U-EtOH suspension were prepared. CCl₄ was dissolved in corn oil at 40% concentration for inducing liver fibrosis in rats. ORC001R-U-EtOH extract was dissolved in a mix solution composed with 1% Tween 80+99% volume of 0.5% CMC (Carboxymethyl cellulose) to create a suspension.

10 SD male rats of 8-10 weeks of age were obtained from Lasco and randomly divided into the following groups: Vehicle group (administered 1% Tween80+99% volume of 0.5% CMC, without CCl₄ induction; n=3), CCl₄ control (n=3), ORC001R-U-EtOH 1 mg/Kg-BW (Kg body weight) (n=2), and ORC001R-U-EtOH 10 mg/Kg-BW (n=2).

All groups were daily administered according to their respective treatment or 1% Tween80+99% volume of 0.5% CMC (Vehicle group) orally for one week before liver fibrosis induction by 40% CCl₄, and then kept the same administration steps daily for 8 weeks. The animals were induced liver fibrosis through intraperitoneal (I.P.) injection twice a week for a total duration of 8 weeks. On the day of liver fibrosis induction, the animals were administered one hour after intraperitoneal injection of CCl₄.

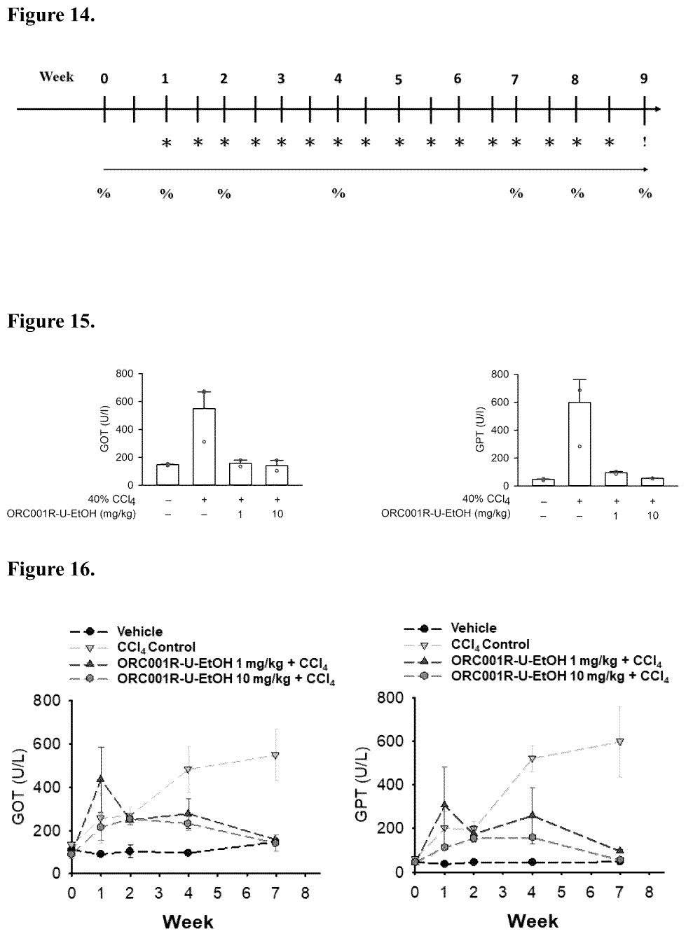

Blood samples were collected at week 0, 1, 2, 4, 7, 8 and 9 for biochemical analysis of GOT, GPT, Albumin, Triglycerides, and Cholesterol. At the end of 9 weeks, all rats were humanely sacrificed; and blood, liver, kidney, and spleen samples were taken. The timeline of the experiment is shown in FIG. 14.

The results of week 7 blood biochemical analysis, weekly changes in blood biochemical analysis and rats body weight tracking are shown in FIGS. 15-17.

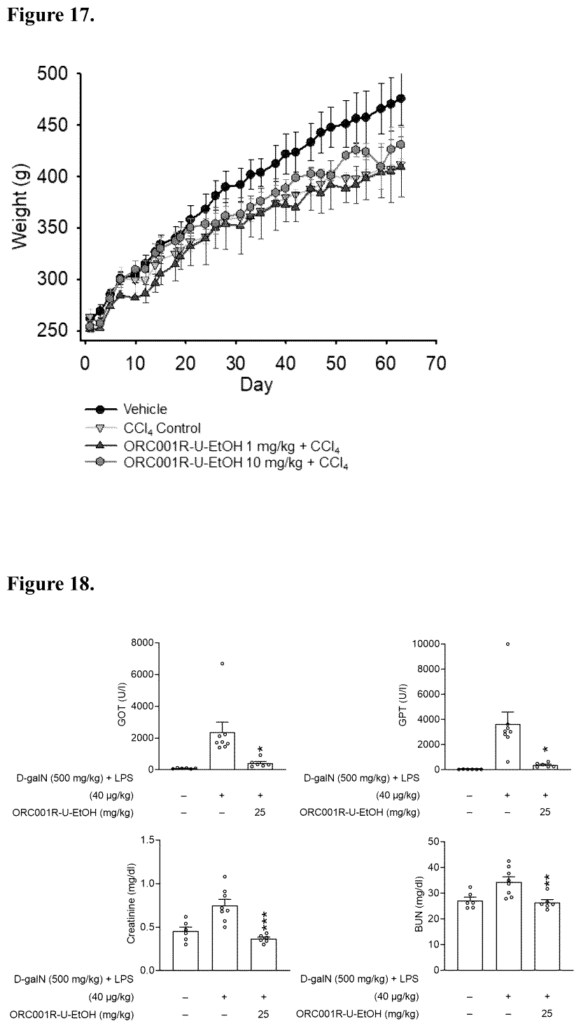

The result of week 7 blood biochemical analysis (FIG. 15) and the result of weekly changes in blood biochemical analysis (FIG. 16) show that both low dose (1 mg/Kg-BW) and high dose of (10 mg/Kg-BW) ORC001R-U-EtOH had remarkably reduction of serum GOT and GPT levels, which indicated that the ORC001R-U-EtOH had the potential in liver protection. The results of the weight tracking of the rats (FIG. 17) showed that the ORC001R-U-EtOH of the present invention did not cause adverse effects to the rats.

Another experiment that combined treatment of D-GalN (D-galactosamine) and LPS induced actue liver injury (ALI) in mice.

Male C57BL/6 mice were randomly divided into three groups (6-8 mice/group): vehicle alone (DMSO, wihtout LPS+D-GalN, without ORC001R-U-EtOH), LPS+D-GalN control (LPS, 40 μg/mL; D-galactosamine, 500 mg/Kg, without ORC001R-U-EtOH), LPS+D-GalN+ORC001R-U-EtOH extract (25 mg/Kg-body weight) group. Each mouse was intraperitoneally injected with 10% DMSO or LPS+D-GalN for 1 hr, followed by intravenous injection of the ORC001R-U-EtOH extract to the corresponding group for another 5 hrs. Blood samples were collected and analyzed for levels of glutamate pyruvate transaminase (GPT), glutamate oxaloacetic transaminase (GOT), creatinine (CRE), and urea nitrogen (BUN).

The results showed that D-GalN/LPS caused significant liver tissue damage, as evidenced by elevated levels of GOT, GPT, CRE, and BUN in the sera. However, treatment with the Oncidium plant extract effectively reduced the elevated levels of GOT and GPT (FIG. 18).

Previous study has shown that Oncidium sotoanum, a species of Oncidium plant, comprises Chrysin (5,7 dihydroxyflavone), which has the effect for liver protection or improvement of diabetes mellitus. The present invention therefore analyzed whether the Oncidium plant extract of the present invention comprised Chrysin. Interestingly, there was no chemical signal of Chrysin detected in the Oncidium plant extract of the present invention (data not shown).

### Evaluating the Effect for Preventing Acute Respiratory Distress Syndrome (ARDS)

All experiments on animals complied with Chang Gung University's IACUC guidelines.

15 BALB/c male mouse with 7-10 weeks of age were selected and randomly divided into the following groups: negative control group (untreated, uninduced; n=3), Lipopolysaccharides (LPS) induced control (untreated, ARDS induced by LPS; n=3), low-dosage group (administered 5 mg/kg body weight ORC001R-U-EtOH, ARDS induced by LPS; n=3), middle-dosage group (administered 10 mg/kg body weight ORC001R-U-EtOH, ARDS induced by LPS; n=3), and high-dosage group (administered 20 mg/kg body weight ORC001R-U-EtOH, ARDS induced by LPS; n=3).

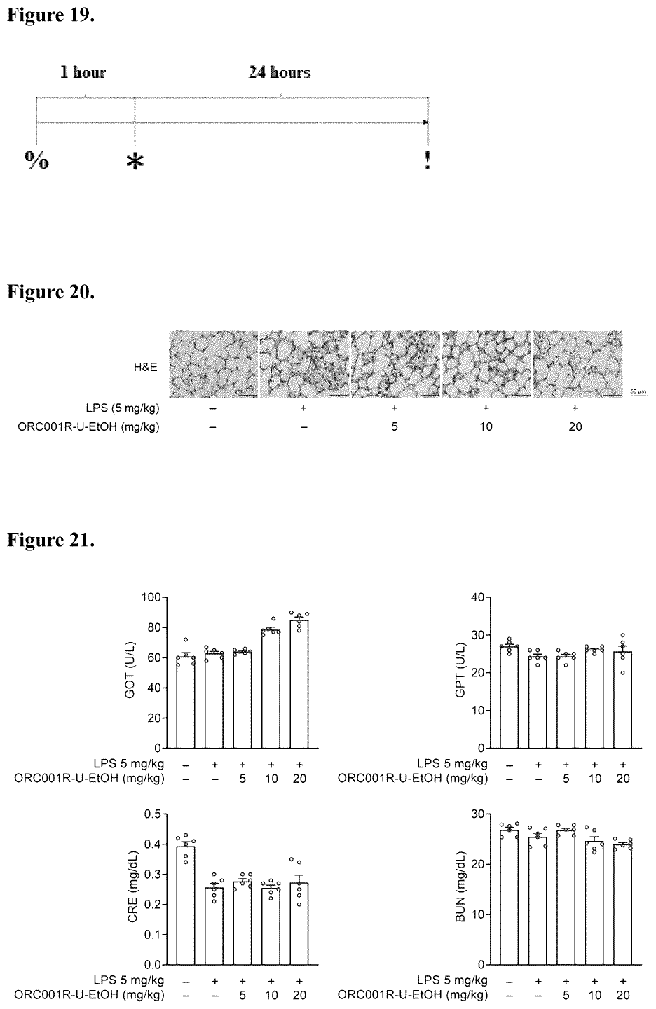

As shown in FIG. 19, at beginning of the experiment, except for the negative control group and the LPS control group, all experimental animals were first given intravenous injection (I.V.) of ORC001R-U-EtOH of the present invention according to the groups, respectively. One hour after ORC001R-U-EtOH administration, all experimental animals except the negative control group were administered 5 mg/kg body weight of LPS-induced ARDS in mice using a lung administration sprayer (purchased from Shanghai Yuyan Instruments Co., Ltd.). All experimental animals were euthanized 24 hours after ARDS induction, blood was collected, centrifuged, the supernatant was taken for subsequent biochemical value detection, and lungs were collected under 21 cm H₂O column pressure, half of which was used for hematoxylin-eosin (H&E) tissue section staining analysis, and the other half was frozen at −80° C. for later use.

The results are shown in FIG. 20 and FIG. 21, staining of lung tissue sections showed that the administration of ORC001R-U-EtOH of the present invention could effectively prevent the occurrence of ARDS or alleviate the degree of ARDS, and it was dose-dependent. The blood analysis results also show that the ORC001R-U-EtOH of the present invention had no obvious harm to the liver and kidney of experimental animals.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cells, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. An Oncidium plant extract, which is prepared by the following steps:
taking an Oncidium plant sample;
drying and grinding the sample to obtain a ground Oncidium plant sample; and
extracting the ground Oncidium plant sample in a solvent by an accelerated solvent extraction or an ultrasonic extraction to obtain an Oncidium plant extract.

2. The Oncidium plant extract of claim 1, wherein the solvent is alcohols, esters, or water, and wherein the Oncidium plant extract comprises alcohols extract, esters extract, or an extraction that is extracted by water and then extracted by esters of the Oncidium plant.

3. The Oncidium plant extract of claim 1, wherein the Oncidium plant extract is extracted from the root, the stem or the leaf of the Oncidium plant, or the mixture composed of the root, the stem and the leaf of the Oncidium plant.

4. The Oncidium plant extract of claim 3, wherein the Oncidium plant extract is extracted from the root of the Oncidium plant.

5. The Oncidium plant extract of claim 1, wherein the species of Oncidium comprises Oncidium stacyi, Oncidium splendidum, Oncidium pusillum, Oncidium jonesianum, Oncidium hawkesiana, Oncidium ascendens, Oncidium ampliatum, or Oncidium flexuosum.

6. A method for treating diabetes mellitus, comprising administering an Oncidium plant extract to a subject suffering from diabetes mellitus.

7. The method of claim 6, wherein the Oncidium plant extract is extracted by the following steps:
taking an Oncidium plant sample;
drying and grinding the sample to obtain a ground Oncidium plant sample; and
extracting the ground Oncidium plant sample in a solvent by an accelerated solvent extraction or an ultrasonic extraction to obtain an Oncidium plant extract.

8. The method of claim 7, wherein the solvent is alcohols, esters, or water, and wherein the Oncidium plant extract comprises alcohols extract, esters extract, or an extraction that is extracted by water and then extracted by esters of the Oncidium plant.

9. The method of claim 6, wherein an effective amount of the Oncidium plant extract is 0.007 mg/kg-body weight to 100 mg/kg-body weight.

10. The method of claim 6, wherein an effective amount of the Oncidium plant extract is 0.035 mg/kg-body weight to 75 mg/kg-body weight.

11. The method of claim 6, wherein an effective amount of the Oncidium plant extract in human is 0.07 mg/kg-body weight to 50 mg/kg-body weight.

12. The method of claim 6, wherein the Oncidium plant extract is extracted from the root, the stem or the leaf of the Oncidium plant, or the mixture composed of the root, the stem and the leaf of the Oncidium plant.

13. The method of claim 12, wherein the Oncidium plant extract is extracted from the root of the Oncidium plant.

14. The method of claim 6, wherein the species of Oncidium comprises Oncidium stacyi, Oncidium splendidum, Oncidium pusillum, Oncidium jonesianum, Oncidium hawkesiana, Oncidium ascendens, Oncidium ampliatum, or Oncidium flexuosum.

15. The method of claim 6, wherein the subject is mammal or human.

16. A method for liver protection, comprising administering an Oncidium plant extract to a subject suffering in need thereof.

17. The method of claim 16, wherein the Oncidium plant extract is extracted by the following steps:
taking an Oncidium plant sample;
drying and grinding the sample to obtain a ground Oncidium plant sample; and
extracting the ground Oncidium plant sample in a solvent by an accelerated solvent extraction or an ultrasonic extraction to obtain an Oncidium plant extract.

18. The method of claim 17, wherein the solvent is alcohols, esters, or water, and wherein the Oncidium plant extract comprises alcohols extract, esters extract, or an extraction that is extracted by water and then extracted by esters of the Oncidium plant.

19. The method of claim 16, wherein an effective amount of the Oncidium plant extract is 0.001 mg/kg-body weight to 50 mg/kg-body weight.

20. The method of claim 16, wherein an effective amount of the Oncidium plant extract is 0.009 mg/kg-body weight to 25 mg/kg-body weight.

21. The method of claim 16, wherein an effective amount of the Oncidium plant extract is 0.015 mg/kg-body weight to 10 mg/kg-body weight.

22. The method of claim 16, wherein the Oncidium plant extract is extracted from the root, the stem or the leaf of the Oncidium plant, or the mixture composed of the root, the stem and the leaf of the Oncidium plant.

23. The method of claim 22, wherein the Oncidium plant extract is extracted from the root of the Oncidium plant.

24. The method of claim 16, wherein the species of Oncidium comprises Oncidium stacyi, Oncidium splendidum, Oncidium pusillum, Oncidium jonesianum, Oncidium hawkesiana, Oncidium ascendens, Oncidium ampliatum, or Oncidium flexuosum.

25. The method of claim 16, wherein the subject is mammal or human.

26. A method for preventing acute respiratory distress syndrome (ARDS), comprising administering an Oncidium plant extract to a subject suffering from ARDS.

27. The method of claim 26, wherein the Oncidium plant extract is extracted by the following steps:
taking an Oncidium plant sample;
drying and grinding the sample to obtain a ground Oncidium plant sample; and
extracting the ground Oncidium plant sample in a solvent by an accelerated solvent extraction or an ultrasonic extraction to obtain an Oncidium plant extract.

28. The method of claim 27, wherein the solvent is alcohols, esters, or water, and wherein the Oncidium plant extract comprises alcohols extract, esters extract, or an extraction that is extracted by water and then extracted by esters of the Oncidium plant.

29. The method of claim 26, wherein an effective amount of the Oncidium plant extract in human is 0.007 mg/kg-body weight to 50 mg/kg-body weight.

30. The method of claim 26, wherein an effective amount of the Oncidium plant extract in human is 0.02 mg/kg-body weight to 25 mg/kg-body weight.

31. The method of claim 26, wherein an effective amount of the Oncidium plant extract in human is 0.035 mg/kg-body weight to 20 mg/kg-body weight.

32. The method of claim 26, wherein the Oncidium plant extract is extracted from the root, the stem or the leaf of the Oncidium plant, or the mixture composed of the root, the stem and the leaf of the Oncidium plant.

33. The method of claim 32, wherein the Oncidium plant extract is extracted from the root of the Oncidium plant.

34. The method of claim 26, wherein the species of Oncidium comprises Oncidium stacyi, Oncidium splendidum, Oncidium pusillum, Oncidium jonesianum, Oncidium hawkesiana, Oncidium ascendens, Oncidium ampliatum, or Oncidium flexuosum.

35. The method of claim 26, wherein the subject is mammal or human.
